(19) United States
(12) Patent Application Publication
(10) Pub. No.: US 2026/0207381 A1
(43) Pub. Date: Jul. 23, 2026

(54) SYSTEM FOR CUTTING OCULAR TISSUE INTO ELEMENTARY PORTIONS

(71) Applicant: KERANOVA, SAINT ETIENNE (FR)

(72) Inventors: Emmanuel BAUBEAU, SAINT ETIENNE (FR); Aurelien BERNARD, SAINT ETIENNE (FR)

(21) Appl. No.: 19/138,027

(22) PCT Filed: Dec. 12, 2023
(86) PCT No.: PCT/EP2023/085289

§ 371 Date: Jun. 11, 2025

(30) Foreign Application Priority Data
Dec. 12, 2022 (FR) FR2213179

(51) Int. Cl. A61F 9/008; A61B 18/00

(52) U.S. Cl. CPC A61F 9/008; A61B 2018/00601; A61F 2009/0087; A61F 2009/00897

(57) ABSTRACT

The invention relates to a cutting device including a femtosecond laser source for emitting a Gaussian laser beam, a shaping system including a spatial light modulator for modulating the Gaussian laser beam, an optical scanner for moving the modulated laser beam, an optical focusing system for focusing the modulated laser beam, characterised in that the treatment device also comprises a control unit for controlling the femtosecond laser source, the shaping system, the optical scanner and the optical focusing system in order to create first and second sets of vertical cutting planes (V1, V2) that are separated from one another by a horizontal cutting plane (H2), the vertical planes (V2) of the second set being offset laterally relative to the vertical planes (V1) of the first set.

### TECHNICAL FIELD

The present invention relates to the technical field of surgical operations performed with femtosecond laser, and more particularly to the field of ophthalmic surgery, in particular for applications of cutting corneas or crystalline lenses.

The invention relates to a device for cutting human or animal tissue, such as a cornea or a crystalline lens, by means of a femtosecond laser source.

By “femtosecond laser source” is meant a light source able to emit a laser beam in the form of ultra-short pulses, the duration of which is comprised between 1 femtosecond and 100 picoseconds, preferably comprised between 1 and 1,000 femtoseconds, in particular of the order of a hundred femtoseconds.

### PRIOR ART

As part of some surgeries, such as cataract surgery, it is desirable to subdivide a tissue such as the crystalline lens C, into small particles such as cubes 108 to facilitate its extraction for example at the area of a suction cannula CA, as illustrated in FIG. 1.

To partition the crystalline lens into cubes 108 that can be suctioned by a suction cannula, horizontal 104a, 104b and vertical 107 cutting planes can be formed, as illustrated in FIG. 2. These plans are formed by starting with the deepest horizontal cutting plane 104a in the crystalline lens and by stacking the successive vertical 107 and horizontal 104b cutting planes up to the most superficial horizontal cutting plane in the crystalline lens.

Document WO 2022/090408 describes in particular a cutting apparatus that allows the creation of horizontal and vertical cutting planes. This cutting apparatus comprises:
a femtosecond laser source to emit a Gaussian laser beam in the form of pulses,
a shaping system positioned downstream of the laser source to modulate the phase of the wavefront of the Gaussian laser beam, the shaping system comprising a spatial light modulator (SLM) and being configured to produce a modulated laser beam from the Gaussian laser beam,
a sweeping optical scanner disposed downstream of the shaping system to move the modulated laser beam,
an optical focusing system downstream of the sweeping optical scanner, to focus the modulated laser beam in a focal plane of the cutting apparatus and to move the focal plane into a plurality of positions along an optical axis of propagation of the modulated laser beam,
a control unit configured to drive the femtosecond laser source, the shaping system, the sweeping optical scanner and the optical focusing system, in order to create successive horizontal and vertical cutting planes.

Referring to FIG. 3, the operating principle of such a cutting apparatus is as follows.

In a first step, an initial horizontal cutting plane 104a (i.e. the deepest) is created. The control unit:
applies a multipoint phase mask to the shaping system to produce a multipoint modulated laser beam that allows simultaneously generating a plurality of impact points, controls the movement of the focusing system to make the focal plane of the cutting apparatus coincide with the desired initial cutting plane,
activates the femtosecond laser source, and
drives the movement of the sweeping optical scanner along the optical path (for example in crenellation).

A series of shots is performed in the focal plane of the cutting apparatus. With each shot, several impact points—forming a pattern—simultaneously focus in the focal plane. Each impact point forms a gas bubble. The optical scanner allows moving the multipoint modulated laser beam—and therefore the pattern—in the focal plane between each shot. When the entire surface of the horizontal cutting plane is covered with gas bubbles, the initial horizontal cutting plane 104a is finalized.

In a second step, several adjacent vertical cutting planes 107a are created. For each vertical cutting plane, the control unit:
applies a conical phase mask (i.e., that allows applying a linear phase modulation with rotational symmetry) to the shaping system to produce a Bessel modulated laser beam,
controls the movement of the focusing system to focus the modulated laser beam to a desired depth,
activates the femtosecond laser source, and
drives the movement of the sweeping optical scanner along the optical path (for example a segment).

A series of shots is performed. With each shot, a line of impact is formed. Each line of impact generates an oblong gas bubble along the optical axis of propagation of the modulated laser beam. The optical scanner allows moving the modulated laser beam—and therefore the line of impact—between each shot. If the depth of the line of impact is smaller than the desired depth for the vertical cutting plane, then the control unit 60 can monitor the optical focusing system 50 to vary the depth of the focal plane of the cutting apparatus.

The vertical cutting planes 107a are completed when the entire movement path is covered with oblong gas bubbles.

The steps of creating horizontal 104b, 104c, 104d and vertical 107b, 107c planes are repeated to create a stack of crystalline cubes 108.

When the multitude of gas bubbles has been formed in the different horizontal and vertical planes, the crystalline cubes thus formed can be separated from the uncut portions of tissue by detaching the existing tissue bridges between the gas bubbles using a tool.

However, the formation of a stack of cubes 108 induces the accumulation of gas in the upper part of the stack of planes, as illustrated in FIG. 4. More specifically, as the stacked cubes 108 are formed, the gas bubbles 109 formed in the depth of the tissue 2 move towards the most superficial region of the tissue.

This gas accumulation can rise above the laser cutting plane into the tissue—for example into the anterior chamber of the eye when cutting the crystalline lens. This can cause problems of penetration of the laser beam into the tissue. Indeed, the gas bubbles form an opaque bubble barrier preventing the propagation of the energy derived from the laser beam beneath them, and thus the cutting of the tissue in the regions located beneath this gas accumulation.

This gas accumulation can also cause a tissue deformation during the cutting which can lead to a defect in the dimensioning of the cubes making their suction from a cannula difficult.

One aim of the present invention is to propose a device for cutting a human or animal tissue that allows preventing the formation of a gas accumulation that could impede the propagation of the laser beam energy beneath said accumulation.

More specifically, one aim of the present invention is to propose a cutting device that allows performing a complete cutting of a tissue—in particular an ocular tissue such as a cornea or a crystalline lens—while avoiding the masking of the laser beam during cutting.

Another aim of the present invention is to propose a device for cutting a human or animal tissue that allows forming tissue cubes of more homogeneous sizes.

### DISCLOSURE OF THE INVENTION

To this end, the invention proposes an apparatus for cutting a human or animal tissue, said apparatus including a femtosecond laser source configured to emit a Gaussian laser beam in the form of pulses and a device for processing the Gaussian laser beam, the processing device being disposed downstream of the femtosecond laser source, the processing device comprising:
a shaping system positioned on the trajectory of the Gaussian laser beam, to modulate the phase of the wavefront of the Gaussian laser beam, the shaping system comprising a spatial light modulator and being configured to produce a modulated laser beam from the Gaussian laser beam,
a sweeping optical scanner disposed downstream of the shaping system to move the modulated laser beam,
an optical focusing system downstream of the shaping system, to focus the modulated laser beam in a focal plane of the cutting apparatus and to move the focal plane of the cutting apparatus into a plurality of positions along an optical axis of propagation of the modulated laser beam,
   remarkable in that the processing device further comprises a control unit to drive the femtosecond laser source, the shaping system, the sweeping optical scanner and the optical focusing system in order to create successive horizontal and vertical cutting planes, the horizontal cutting planes extending perpendicularly to the optical axis and the vertical cutting planes extending parallel to the optical axis, said control unit being configured to:
control the creation of a first horizontal cutting plane,
control the creation of a first plurality of vertical cutting planes above the first horizontal cutting plane,
control the creation of a second horizontal cutting plane above the first plurality of vertical cutting planes, the first horizontal cutting plane being deeper in the tissue than the second horizontal cutting plane,
control the creation of a second plurality of vertical planes above the second horizontal plane, in which the second plurality of vertical cutting planes is laterally offset (along at least one direction perpendicular to the optical axis) relative to the first plurality of vertical planes.

Within the framework of the present invention, by “horizontal cutting plane” is meant a plane located in the tissue to be treated and extending perpendicularly to the optical axis of propagation of the laser beam derived from the cutting apparatus.

Within the framework of the present invention, by “vertical cutting plane” is meant a plane located in the tissue to be treated and extending parallel to an optical axis of propagation of the laser beam derived from the cutting apparatus.

Within the framework of the present invention, by “impact point” is meant a point zone of the laser beam comprised in the focal plane of the cutting apparatus and in which the intensity of the laser beam is sufficient to generate a gas bubble in a tissue.

Within the framework of the present invention, by “line of impact” is meant a linear zone of the laser beam extending perpendicularly to the focal plane of the cutting apparatus (i.e., a segment of the laser beam extending parallel to the optical axis) and in which the intensity of said laser beam is sufficient to generate an oblong gas bubble in the tissue.

Within the framework of the present invention, by “adjacent impact points” is meant two impact points disposed facing each other and not separated by another impact point.

By “neighboring impact points” is meant two points in a group of adjacent points between which the distance is minimal.

Within the framework of the present invention, by “pattern” is meant a plurality of laser impact points generated simultaneously.

Thus, the invention allows generating several groups of vertical cutting planes separated by horizontal cutting planes to form elementary cubes of tissues that can be suctioned by a suction cannula. Advantageously, each group of vertical cutting planes is laterally offset relative to the groups of vertical cutting planes adjacent thereto.

Particularly, each group is defined by a plurality of vertical cutting planes formed between two horizontal cutting planes. In the event that three superimposed horizontal cutting planes are arranged in the tissue, namely:
a deep horizontal cutting plane,
an intermediate horizontal cutting plane, and
a superficial horizontal cutting plane,
   then two groups of vertical cutting planes are formed between the horizontal cutting planes:
a first group of vertical cutting planes is formed between the deep horizontal cutting plane and the intermediate horizontal cutting plane, and
a second group of vertical planes is formed between the intermediate cutting plane and the superficial cutting plane, the vertical cutting planes of the second group being laterally offset relative to the vertical cutting planes of the first group.

This lateral offset between vertical cutting planes of adjacent group allows preventing the gas contained in the gas bubbles formed in one group of vertical cutting planes from propagating towards the gas bubbles formed in a more superficial adjacent group of vertical cutting planes, as will be apparent from the following description.

The optical phase modulation is performed by means of a phase mask. The energy of the incident laser beam is conserved after modulation, and the beam shaping is performed by acting on its wavefront. The phase of an electromagnetic wave represents the instantaneous situation of the amplitude of an electromagnetic wave. The phase depends on both time and space. In the case of spatial shaping of a laser beam, only the variations in the space of the phase are considered.

The wavefront is defined as the surface of the points on a beam with an equivalent phase (i.e., the surface consisting of the points whose travel times from the source that emitted the beam are equal). The modification of the spatial phase of a beam therefore involves the modification of its wavefront.
Within the framework of the present invention, the phase modulation of the wavefront allows generating a single modulated laser beam that forms:
either several impact points only in the cutting plane (used to form a horizontal cutting plane); in this case, the modulated laser beam is unique throughout the propagation path, the phase modulation of the wavefront making it possible to delay or advance the phase of the different points of the surface of the beam relative to the initial wavefront so that each of these points creates constructive interference at N distinct points in the focal plane of a lens, the redistribution of energy into a plurality of impact points taking place only in a single plane (i.e. the focusing plane) and not throughout the propagation path of the modulated laser beam,
or a line of impact perpendicular to the focal plane of the cutting apparatus (used to form a vertical cutting plane).

Preferred but not limiting aspects of the cutting apparatus are the following:
the second plurality of vertical cutting planes can be laterally offset relative to the first plurality of vertical cutting planes by a distance comprised between 5 μm and 500 μm;
the second plurality of vertical cutting planes can be laterally offset relative to the first plurality of vertical cutting planes along first and second axes perpendicular to the optical axis, the first and second axes being orthogonal to each other;
for the creation of each horizontal cutting plane, the control unit can be configured to:
   apply a multipoint phase mask to the shaping system to produce a single multipoint modulated laser beam, the multipoint phase mask being calculated to distribute the energy of the multipoint modulated laser beam into at least two impact points in the focal plane of the cutting apparatus,
   control the movement of the focusing system to make the focal plane of the cutting apparatus coincide with the desired depth for the horizontal cutting plane, drive the sweeping optical scanner to move the impact points of the single multipoint modulated laser beam along a first movement path,
   activate the femtosecond laser source;
for the creation of each vertical cutting plane of the first plurality, the control unit can be configured to:
   apply an axicon modulation setpoint to the shaping system in order to produce a Bessel-type modulated laser beam from the Gaussian laser beam, said modulation setpoint including a phase mask emulating an axicon applied to the spatial light modulator, said Bessel-type modulated laser beam creating a line of impact that allows generating an oblong gas bubble in the tissue,
   drive the sweeping optical scanner to move the line of impact of the Bessel-type modulated laser beam along a second optical movement path to form a set of adjacent oblong gas bubbles;
for the creation of each vertical cutting plane of the second plurality, the control unit is configured to:
   apply the axicon modulation setpoint to the shaping system,
   drive the sweeping optical scanner to move the line of impact of the Bessel-type modulated laser beam along a third optical path laterally offset relative to the second optical path;
each vertical cutting plane is composed of a stack of several sets of adjacent oblong gas bubbles, the control unit (60) being configured to:
   drive the optical focusing system in order to position the focal plane of the cutting apparatus at a predefined non-zero distance from the first horizontal cutting plane, said predefined distance being smaller than the length of the line of impact of the Bessel-type modulated laser beam such that the line of impact partially intersects the first horizontal cutting plane,
   drive the sweeping optical scanner to move the line of impact of the Bessel-type modulated laser beam to form a first set of adjacent oblong gas bubbles,
   drive the optical focusing system in order to position the focal plane of the cutting apparatus at the predefined distance from the first set of adjacent oblong gas bubbles such that the line of impact partially intersects the first set of adjacent oblong gas bubbles,
   drive the sweeping optical scanner to move the line of impact of the Bessel-type modulated laser beam to form a second set of adjacent oblong gas bubbles;
the predefined distance can be comprised between ⅕ and ⅓ of the length of the line of impact.

The invention also relates to a method for cutting a tissue, for example a previously collected human or animal tissue, from a cutting apparatus including:
a femtosecond laser source configured to emit a Gaussian laser beam in the form of pulses,
a shaping system downstream of the femtosecond laser source and positioned on the trajectory of the Gaussian laser beam, to modulate the phase of the wavefront of the Gaussian laser beam, the shaping system comprising a spatial light modulator and being configured to produce a modulated laser beam from the Gaussian laser beam,
a sweeping optical scanner disposed downstream of the shaping system to move the modulated laser beam,
an optical focusing system downstream of the shaping system, to focus the modulated laser beam in a focal plane of the cutting apparatus and to move the focal plane of the cutting apparatus into a plurality of positions along an optical axis of propagation of the modulated laser beam,
   remarkable in that the cutting method comprises a phase of creating successive horizontal and vertical cutting planes by driving the femtosecond laser source, the shaping system, the sweeping optical scanner and the optical focusing system, the horizontal cutting planes extending perpendicularly to the optical axis and the vertical cutting planes extending parallel to the optical axis, said creation phase comprising the steps consisting in:
forming a first horizontal cutting plane,
forming, above the first horizontal cutting plane, a first plurality of vertical cutting planes,
forming, above the first plurality of vertical cutting planes, a second horizontal cutting plane, the first horizontal cutting plane being deeper in the tissue than the second horizontal cutting plane,
forming, above the second horizontal plane, a second plurality of vertical planes laterally offset relative to the first plurality of vertical planes.

Preferred but non-limiting aspects of the method according to the invention are the following:
the second plurality of vertical cutting planes may be laterally offset relative to the first plurality of vertical cutting planes by a distance comprised between 5 μm and 500 μm;
the second plurality of vertical cutting planes may be laterally offset relative to the first plurality of vertical cutting planes along first and second axes perpendicular to the optical axis, the first and second axes being orthogonal to each other;
each step consisting in forming a horizontal cutting plane may comprise the following sub-steps:
   applying a multipoint phase mask to the shaping system to produce a single multipoint modulated laser beam, the multipoint phase mask being calculated to distribute the energy of the multipoint modulated laser beam into at least two impact points in the focal plane of the cutting apparatus,
   moving with the focusing system the focal plane of the cutting apparatus to the desired depth for the horizontal cutting plane,
   moving with the sweeping optical scanner the impact points of the single multipoint modulated laser beam along a first movement path, and
   emitting a Gaussian laser beam by the femtosecond laser source;
each step consisting in forming a vertical cutting plane of the first plurality may comprise the following sub-steps:
   applying an axicon modulation setpoint to the shaping system in order to produce a Bessel-type modulated laser beam, said modulation setpoint including a phase mask emulating an axicon applied to the spatial light modulator, said phase mask having a symmetry of revolution around a central point of symmetry, the grayscale of each point of the phase mask varying as a function of the distance between said point and the central point of symmetry, said Bessel-type modulated laser beam creating a line of impact that allows generating an oblong gas bubble in the tissue,
   moving with the sweeping optical scanner the line of impact of the Bessel-type modulated laser beam along a second optical movement path to form a set of adjacent oblong gas bubbles;
each step consisting in forming a vertical cutting plane of the second plurality may comprise the following sub-steps:
   applying the axicon modulation setpoint to the shaping system,
   moving with the sweeping optical scanner the line of impact of the Bessel-type modulated laser beam along a third optical movement path laterally offset relative to the second optical path;
each vertical cutting plane may be composed of a stack of several sets of adjacent oblong gas bubbles, each step consisting in forming a vertical cutting plane comprising the following sub-steps:
   moving with the optical focusing system the focal plane of the cutting apparatus at a predefined non-zero distance from the first horizontal cutting plane, said predefined distance being smaller than the length of the line of impact of the Bessel-type modulated laser beam so that the line of impact partially intersects the first horizontal cutting plane,
   moving with the sweeping optical scanner the line of impact of the Bessel-type modulated laser beam to form a first set of adjacent oblong gas bubbles,
   moving with the optical focusing system the focal plane of the cutting apparatus at the predefined distance from the first set of adjacent oblong gas bubbles so that the line of impact partially intersects the first set of adjacent oblong gas bubbles,
   moving with the sweeping optical scanner the line of impact of the Bessel-type modulated laser beam to form a second set of adjacent oblong gas bubbles;
the predefined distance can be comprised between ⅕ and ⅓ of the length of the line of impact.

### BRIEF DESCRIPTION OF THE DRAWINGS

Other characteristics and advantages of the invention will emerge clearly from the description given below, for information and without limitation, with reference to the appended figures, in which:

FIG. 1 is a schematic representation of a patient's eye,

FIG. 2 is a schematic representation of gas bubbles created to form elementary tissue cubes from horizontal and vertical cutting planes,

FIG. 3 is a schematic representation of a stack of horizontal and vertical cutting planes obtained using the cutting apparatus described in document WO 2016/055539,

FIG. 4 is a schematic representation illustrating an accumulation of gas following the formation of superimposed vertical cutting planes using the cutting apparatus described in document WO 2016/055539,

FIG. 5 is a schematic representation of a cutting apparatus according to the invention,

FIG. 6 is a schematic representation illustrating the focusing of a Bessel-type non-diffracting beam,

FIG. 7a is an image of a first phase mask that allows emulating the behavior of a negative axicon on an SLM of the cutting apparatus according to the invention,

FIG. 7b is an image of a second phase mask that allows emulating the behavior of a positive axicon on the SLM of the cutting apparatus according to the invention,

FIG. 8 is a partial mounting diagram of the cutting device,

FIG. 9 is a schematic representation of a Bessel beam,

FIG. 10 is a schematic representation of control steps during the creation of a horizontal cutting plane,

FIG. 11 is a schematic representation of control steps during the creation of a vertical cutting plane,

FIG. 12 is a schematic representation illustrating the formation of a vertical cutting plane from a Bessel laser beam,

FIG. 13 is a schematic representation illustrating a set of lines of impact used to create stacked oblong gas bubble sections,

FIG. 14 is a schematic representation of control steps during the creation of successive horizontal and vertical cutting planes,

FIG. 15 is a schematic representation of successively created horizontal and vertical cutting planes.

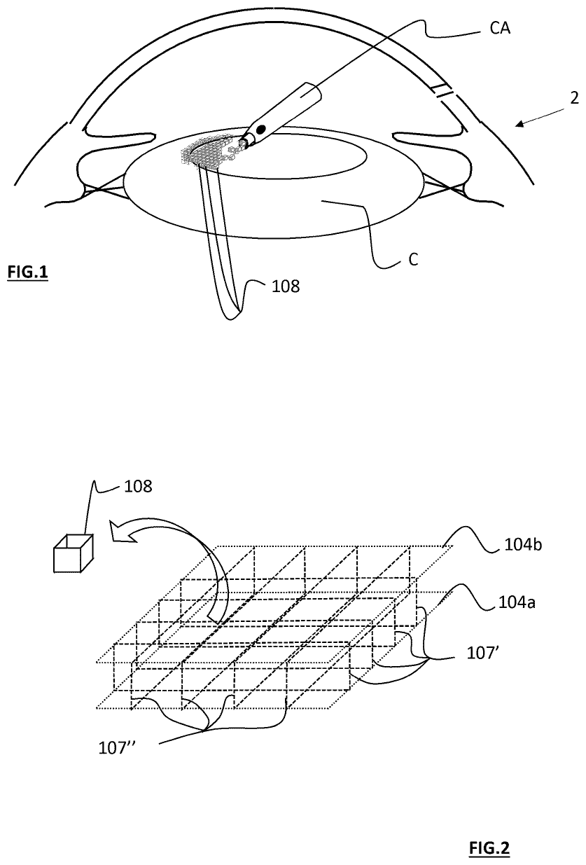

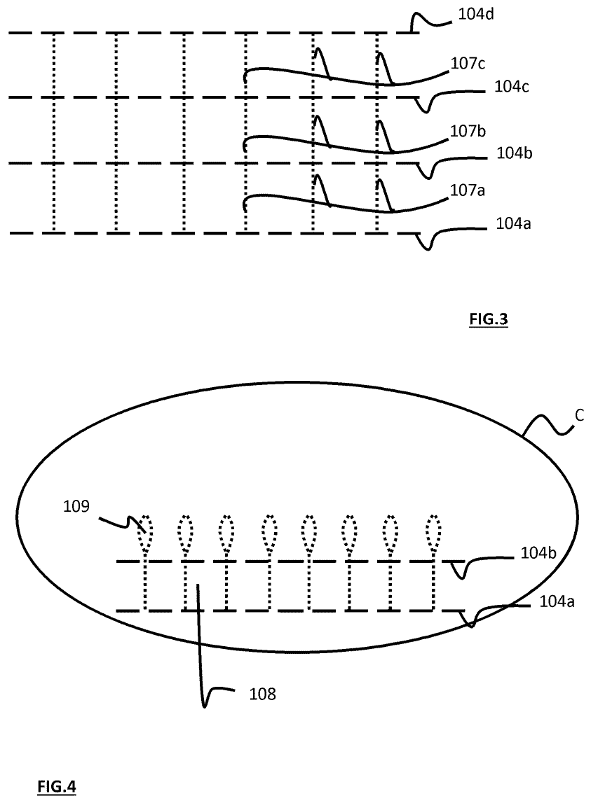

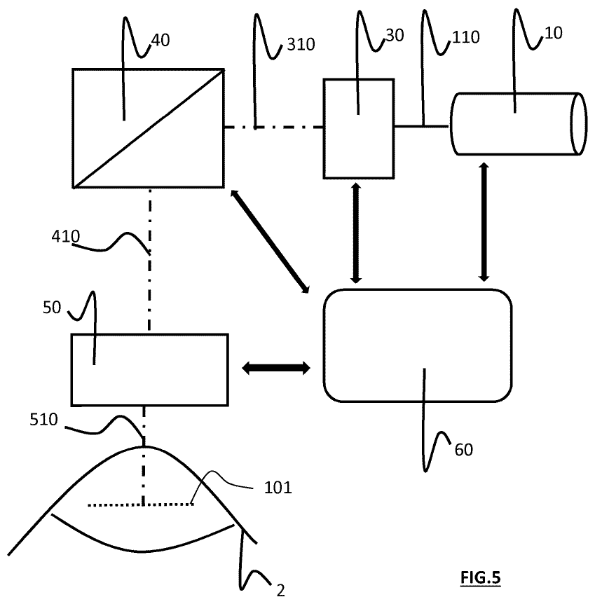

### DETAILED DISCLOSURE OF THE INVENTION

The invention relates to a system for cutting a human tissue by means of a femtosecond laser. In the remainder of the description, the invention will be described, by way of example, for the cutting of a crystalline lens of a human or animal eye.

### 1. CUTTING APPARATUS

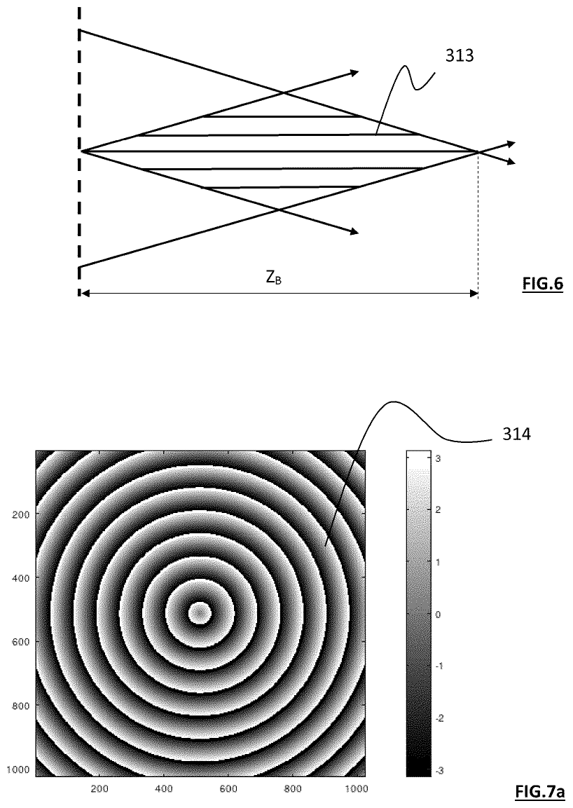

Referring to FIG. 5, one embodiment of the cutting apparatus according to the invention is illustrated. This apparatus can be disposed between a femtosecond laser source 10 and a target 2 to be treated.

The femtosecond laser source 10 is able to emit a Gaussian laser beam in the form of pulses. For example, the femtosecond laser source 10 emits a light with a wavelength of 1,030 nm, in the form of 400 femtosecond pulses. The femtosecond laser source 10 has a power of 20 W and a frequency of 500 KHz.

The target 2 is for example a human or animal tissue to be cut, such as a cornea or a crystalline lens.

The cutting apparatus comprises:
a shaping system 30 positioned on the trajectory of the laser beam 110 derived from the femtosecond laser 10,
a sweeping optical scanner 40 downstream of the shaping system 30,
an optical focusing system 50 downstream of the sweeping optical scanner 40, and
a control unit 60.

The shaping system 30 allows modulating the phase of the laser beam 110 derived from the femtosecond laser source 10. This shaping system 30 is advantageously a programmable component.

The sweeping optical scanner 40 allows orienting the phase-modulated laser beam 310 derived from the shaping system 30 to move the cutting pattern along a user-predefined movement path in the focal plane 101 of the cutting system.

The optical focusing system 50 allows moving the focal plane 101—corresponding to the cutting plane—of the modulated and deflected laser beam 410.

The control unit 60 allows driving the shaping system 30, the sweeping optical scanner 40, and the optical focusing system 50.

This cutting apparatus is adapted to form horizontal and vertical cutting planes. Depending on the type of cutting plane desired (vertical or horizontal), the control unit 60:
configures the shaping system to modulate the laser beam 110 according to the desired appearance for the points/lines of impact, and
monitors the sweeping optical scanner 40 and the optical focusing system 50 to generate the desired cutting plane.

As will be described in more detail below, the inventors have developed an original solution for configuring the cutting apparatus for the formation of vertical cutting planes.

### 2. ELEMENTS OF THE CUTTING APPARATUS

### 2.1. Shaping System

The spatial shaping system 30 of the laser beam allows varying the wave surface of the laser beam 110 according to the desired shape:
for the point(s) of impact of the modulated laser beam in the case of the formation of a horizontal cutting plane, or
for the line(s) of impact of the modulated laser beam in the case of the formation of a vertical cutting plane.

The shaping system 30 preferably comprises a spatial light modulator, known as SLM.

The SLM allows modulating the final energy distribution of the laser beam 110 derived from the laser source 10. The SLM is a device consisting of a layer of liquid crystals with monitored orientation that allows dynamically shaping the wavefront, and therefore the phase of the laser beam 110. The liquid crystal layer of an SLM is organized as a grid (or matrix) of pixels. The optical thickness of each pixel is electrically monitored by orientation of the liquid crystal molecules belonging to the surface corresponding to the pixel. The SLM uses the principle of liquid crystal anisotropy, that is to say the modification of the index of the liquid crystals, depending on their spatial orientation. The orientation of the liquid crystals can be performed using an electric field. Thus, the modification of the index of the liquid crystals modifies the wavefront of the laser beam.

In a known manner, the SLM implements a phase mask, that is to say a map determining how the phase of the laser beam 110 must be modified to obtain a given amplitude distribution. The phase mask is a two-dimensional image, each point of which is associated with a respective pixel of the SLM. This phase mask allows driving the index of each liquid crystal of the SLM by converting the value associated with each point of the mask—represented in grayscale comprised between 0 and 255 (therefore from black to white)—into a control value—represented in a phase comprised between 0 and 2π. Thus, the phase mask is a modulation setpoint displayed on the SLM to cause, upon reflection, an unequal spatial phase shift of the laser beam 110 illuminating the SLM. Of course, those skilled in the art will appreciate that the grayscale range may vary depending on the SLM model used. For example, in some cases, the grayscale range can be comprised between 0 and 220.

Different phase masks can be applied to the SLM depending on the type of cutting plane that the user wishes to create, namely:
either a vertical cutting plane,
or a horizontal cutting plane.

For the creation of a vertical cutting plane, the phase mask used (hereinafter referred to as “conical phase mask”) allows applying a linear phase modulation with rotational symmetry. A Bessel-type modulated laser beam is thus obtained.

For the creation of a horizontal cutting plane, the phase mask used (hereinafter referred to as “multipoint phase mask”) allows applying a phase modulation to distribute the laser beam energy into at least two impact points forming a pattern in the focal plane of the cutting system. A multipoint-type modulated laser beam is thus obtained.

### 2.1.1. Vertical Cutting Plane

With regard to the cutting of a vertical plane, the inventors propose modulating the phase of the laser beam 110 derived from the femtosecond laser source 10 so as to produce, downstream of the shaping system 30, a Bessel-type modulated laser beam 310.

A Bessel beam is called “non-diffracting” beam because it has the property of maintaining a constant profile along the optical axis of propagation of the laser beam (hereinafter referred to as “optical axis”), unlike the behavior of a Gaussian laser beam (such as the laser beam 110 derived from the femtosecond laser source 10) which disperses when it is focused.

### 2.1.1.1. Bessel Beam

A perfect zeroth-order Bessel beam can be defined mathematically as a beam whose electric field € is formally described by the zeroth-order Bessel function of the first kind J₀:

$$E ( r , ϕ , z ) {A}_{0} {J}_{0} ( {k}_{r} r ) {e}_{z}^{jk z}$$

where:
A₀ is the amplitude of the electric field,
k_{z} and kᵣ are the longitudinal and radial wave vectors,
z, r and φ are the longitudinal, radial and azimuthal components.

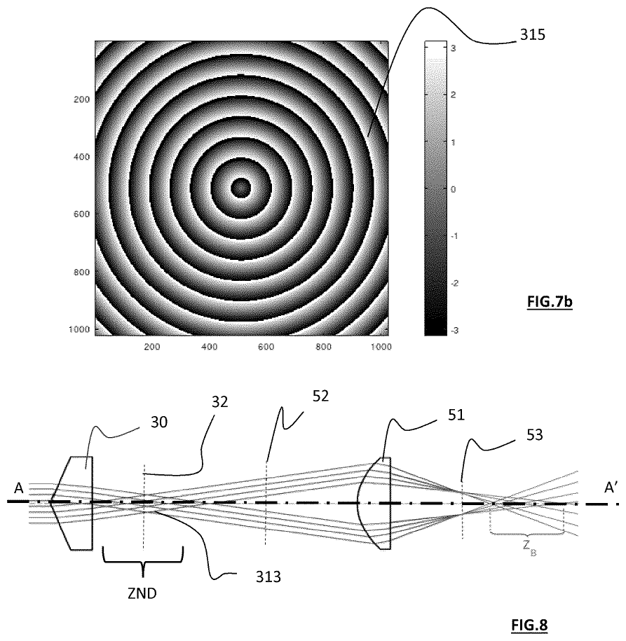

Referring to FIG. 6, the formation of the Bessel beam 313 results from the interference of plane waves whose wave vectors form a conical surface.

In theory, the transverse extension of the annular structure is infinite, as is the non-diffractive propagation distance.

In practice, the experimental Bessel beam has a finite non-diffractive propagation distance Z_{B} along the optical axis due to the finite propagation observed in optics and to the limited amount of energy. This finite non-diffractive propagation distance Z_{B} defines a non-diffraction zone ZND.

It is assumed that Z_{B}>>Z_{R}, Z_{R} being the Rayleigh distance of the usual Gaussian beam of similar transverse size. In other words, the depth (i.e., the dimension along a direction parallel to the optical axis of propagation of the laser beam) of each impact point of a Bessel beam is much greater than the depth of each impact point with a Gaussian laser beam (such as the laser beam derived from the femtosecond laser source).

Thus, the use of a Bessel beam allows cutting a tissue depth much greater than with a Gaussian beam. Particularly, from a single line of impact of a Bessel beam, it is possible to cut a tissue to a depth equivalent to that of four superimposed impact points of a Gaussian beam. The movement, with the sweeping optical scanner, of a line of impact of a Bessel beam allows generating a perfectly vertical cutting plane four times faster than with an impact point of a Gaussian beam.

Due to its specific formation based on a conical wavefront, the Bessel beam has remarkable self-regeneration properties, which means that the beam can regenerate itself within the non-diffraction zone ZND after any obstacle on its path. This ensures the quality of the cutting of the vertical planes by guaranteeing the formation of an extended gas bubble with each shot of the laser source 10, even when part of the modulated laser beam 310 is masked by an obstacle.

### 2.1.1.2. Conical Phase Mask to Form a Bessel-Type Modulated Laser Beam

There are various techniques for generating a Bessel beam from a Gaussian laser beam. These techniques generally involve an axicon phase modulation.

Particularly, the Bessel beam can be obtained by using a conical lens known as “axicon”. The conical lens can be concave/hollow (called “negative axicon”) or convex/bulged (called “positive axicon”).

The inventors propose using the shaping system 30 including the SLM to generate the Bessel beam in order to avoid the use of an optical/mechanical element. To this end, a conical phase mask (that allows emulating an axicon) is applied to the SLM by the control unit 60. The SLM then allows a conical phase modulation of the Gaussian laser beam 110 derived from the femtosecond laser source 10. Thus, by using the same SLM, it becomes possible to create a horizontal cutting plane in multipoints, then vertical cutting planes in Bessel beam modality without changing optical elements and therefore by considerably reducing the surgical procedure time to a time comprised between 30 seconds and 1 minute, compatible with an application on the patient's eyeball of less than 3 minutes.

The conical phase mask to be applied to the SLM of the shaping system to form a Bessel modulated laser beam can be calculated:
by using a partition algorithm (Vellekoop and Mosk, 2008),
or any other algorithm known to those skilled in the art.

Two examples of such phase masks are illustrated in FIGS. 7a and 7b. When one of the first and second phase masks is applied to the SLM, the SLM is capable of imprinting the phase profile of an axicon on the input Gaussian laser beam 110 to obtain a Bessel-type modulated laser beam 310 at the output of the shaping system 30.

Referring to FIG. 7a, the first conical phase mask (referenced 314) allows emulating the behavior of a negative axicon (i.e., concave axicon). Referring to FIG. 7b, the second conical phase mask (referenced 315) allows emulating the behavior of a positive axicon (i.e., convex axicon). These first and second phase masks each have a symmetry of revolution around a central point of symmetry, the grayscale of each pixel varying as a function of the distance between said pixel and the central point of symmetry.

When one of the phase masks illustrated in FIGS. 7a and 7b is applied to the SLM, the shaping system 30 allows forming a Bessel-type modulated laser beam 310 (at the output of the shaping system 30) from the Gaussian laser beam 110 derived from the femtosecond laser source 10 (at the input of the shaping system 30). A modulated laser beam having a spatial intensity distribution in Bessel beam is thus obtained.

2.1.1.3. Mounting the Cutting Apparatus as Part of the Cutting of a Tissue from a Bessel-Type Modulated Laser Beam

FIG. 8 illustrates a cutting apparatus mounting diagram. This mounting diagram is partial in that it does not show the femtosecond laser source and the sweeping optical scanner. Furthermore, in this FIG. 8, the optical focusing system 50 (as a whole) is represented by an equivalent lens 51, it being understood by those skilled in the art that the optical focusing system 50 does not consist solely of a fixed lens.

Referring to FIG. 8, the Bessel beam 313 is formed just after the conical phase modulation plane, that is to say just after the SLM of the shaping system 30. The SLM simulating a conical lens (negative or positive axicon), the central spot of maximum intensity of the Bessel beam is formed in the image focal plane 32 of the SLM.

The equivalent lens 51 of the optical focusing system 50 is disposed downstream of the shaping system 30, and is arranged such that the object focal plane 52 of the equivalent lens extends at a non-zero distance from the image focal plane 32 of the shaping system 30 along the optical axis.

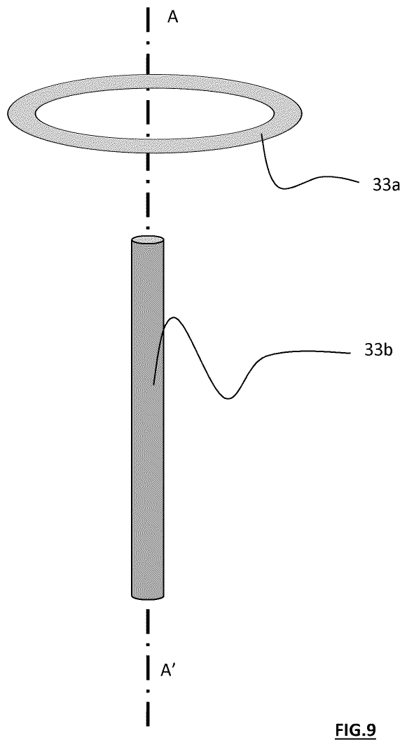

Thus, the object focal plane 52 of the equivalent lens 51 of the optical focusing system 50 extends out of the non-diffraction zone ZND of the Bessel beam, such that at the output of the cutting system, a line of impact as illustrated in FIG. 9 is obtained. More specifically, the Bessel beam is composed:
of a Bessel ring 33a focused on the image focal plane 53 of the equivalent lens 51 (corresponding to the focal plane of the cutting apparatus),
of a line 33b of concentration of the rays of the Bessel beam-corresponding to the image of the non-diffraction zone ZND-said line 33b forming out of the image focal plane 53 of the equivalent lens 51.

Within the framework of the present invention, it is the line 33b that constitutes the line of impact used to create the vertical cutting plane (the energy contained in the Bessel ring is not sufficient to form a gas bubble).

The line 33b can be formed either before or after the ring 33a, depending on the sign of the phase modulation. In other words, the position of the line 33b relative to the ring 33a depends on the type of axicon (positive or negative) emulated using the conical phase mask.

Since the Bessel non-diffraction zone ZND (i.e., the line 33b) is moved out of the focal plane of the cutting system, no interference occurs with the unmodulated light. This allows for better monitoring of the intensity profile without energy loss related to the filtering of the beam.

### 2.1.2. Horizontal Cutting Plane

With regard to the cutting of a horizontal plane, the inventors propose modulating the phase of the laser beam 110 derived from the femtosecond laser source 10 so as to produce, downstream of the shaping system 30, a multipoint-type modulated laser beam.

To this end, a multipoint phase mask to be applied to the SLM to obtain the multipoint modulated laser beam is calculated. The multipoint phase mask is generally calculated by:
an iterative algorithm based on the Fourier transform, such as an IFTA (Iterative Fourier Transform Algorithm) type algorithm, or by
various optimization algorithms, such as genetic algorithms, or the simulated annealing.

This multipoint phase mask is calculated to form intensity peaks in the focal plane of the cutting apparatus, each intensity peak producing a respective impact point in the focal plane of the cutting apparatus.

More specifically, the multipoint phase mask is calculated to distribute the energy of the laser beam derived from the laser source into multiple impact points—forming a pattern—in the focal plane of the cutting apparatus. This wavefront modulation can be viewed as a two-dimensional interference phenomenon. Each portion of the initial laser beam derived from the source is delayed or advanced relative to the initial wavefront so that each of these portions is redirected to create a constructive interference at N distinct points in the focal plane of a lens. This redistribution of energy into a plurality of impact points occurs only in a single plane (i.e., the focusing plane) and not along the entire propagation path of the modulated laser beam. Thus, the multipoint laser beam obtained (at the output of the shaping system 30) is unique: the observation of the modulated laser beam before or after the focal plane of the cutting apparatus (corresponding to the focal plane of the optical focusing system 50) does not allow identifying a redistribution of the energy into a plurality of distinct impact points, due to this phenomenon which can be likened to constructive interference (which only occurs in one plane and not throughout the propagation as in the case of the separation of an initial laser beam into a plurality of secondary laser beams).

Having a single multipoint modulated laser beam facilitates the integration of a sweeping system—such as an optical scanner—to move the plurality of impact points in the focal plane. Indeed, the input diameter of a sweeping system being of the order of the diameter of the initial laser beam derived from the laser source 10, the use of a single multipoint modulated laser beam (whose diameter is substantially equal to the diameter of the initial laser beam) limits the risks of aberration which can occur with the beam subdivision technique as described in document US 2010/0133246.

The shaping system 30 therefore allows, from a Gaussian laser beam generating a single impact point, and by means of the multipoint phase mask applied to the SLM, distributing its energy by phase modulation so as to simultaneously generate several impact points in the focal plane of the cutting apparatus, from a single laser beam shaped by phase modulation (a single beam upstream and downstream of the SLM). This allows reducing the time required to create a horizontal cutting plane.

For example, in the case of a multipoint modulated laser beam having three impact points, the time necessary for the creation of a horizontal cutting plane is reduced by a factor of six (compared to the creation of the same horizontal cutting plane by using a Gaussian laser beam generating a single impact point). Those skilled in the art know how to calculate a value at each point of the multipoint phase mask to distribute the energy of the laser beam at different impact points in the focal plane of the cutting apparatus.

### 2.2. Sweeping Optical Scanner

The sweeping optical scanner 40 allows deflecting the (Bessel or multipoint) modulated laser beam 310 so as to move:
for the creation of a horizontal cutting plane, the point(s) of impact into a plurality of positions along a first movement path,
for the creation of a vertical cutting plane, the line(s) of impact into a plurality of positions along a second movement path.

The sweeping optical scanner 40 comprises:
an input orifice to receive the phase-modulated laser beam 31 derived from the shaping unit 30,
one (or more) optical mirror(s) pivoting around at least two axes to deflect the phase-modulated laser beam 310, and
an output orifice to send the deflected modulated laser beam 410 towards the optical focusing system 50.

The optical scanner 40 used is for example a sweeping head IntelliScan III from the company SCANLAB AG.

The input and output orifices of such an optical scanner 40 have a diameter of around 10 to 20 millimeters, and the achievable sweeping speeds are around 1 m/s to 10 m/s.

The mirror(s) is/are connected to one or more motor(s) to allow their pivoting. This/these motor(s) for the pivoting of the mirror(s) is/are advantageously driven by the control unit 60, which will be described in more detail below.

The control unit 60 is programmed to drive the sweeping optical scanner 40 so as to move:
the point(s) of impact along the first movement path,
the line(s) of impact along the second movement path.

In the case of a horizontal cutting plane, the first movement path comprises a plurality of cutting segments. The first movement path may advantageously have a crenellation shape.

In the case of a vertical cutting plane, the second movement path comprises a segment. The control unit 60 may be configured to command the optical scanner 40 to move the Bessel line of impact back and forth along said segment in order to cut the vertical cutting plane over its entire depth. For example, if the optical scanner 40 starts the segment from the left, it will start this segment from the right on the way back, then from the left, then from the right, and so on over the entire height of the vertical cutting plane.

The sweeping of the beam has an influence on the result of the obtained cutting. Indeed, the used sweeping speed as well as the sweeping pitch are parameters influencing the quality of the cutting.

Advantageously, the control unit 60 can be programmed to activate the femtosecond laser 10 when the sweeping speed of the optical scanner 40 is greater than a threshold value. This allows synchronizing the emission of the laser beam 110 with the sweeping of the sweeping optical scanner 40. More specifically, the control unit 60 activates the femtosecond laser 10 when the pivoting speed of the mirror(s) of the optical scanner 40 is constant. This allows improving the cutting quality by performing homogeneous surfacing of the cutting plane.

### 2.3. Optical Focusing System

The optical focusing system 50 allows moving the focal plane of the cutting apparatus depending on the type of cutting plane to be created.

The optical focusing system 50 comprises:
an input orifice to receive the phase-modulated and deflected laser beam derived from the sweeping optical scanner 40,
one (or more) motorized lens(es) to allow its (their) translational movement along the optical path of the modulated and deflected laser beam, and
an output orifice to send the focused laser beam towards the tissue to be treated.

The control unit 60 is programmed to drive the movement of the lens(es) of the optical focusing system 50 so as to move the focal plane of the cutting apparatus depending on the type of cutting plane to be created.

In the case of a horizontal cutting plane, the cutting plane corresponds to the focal plane of the cutting apparatus. The control unit 60 drives the movement of the lens(es) of the optical focusing system 50 to focus the modulated and deflected laser beam 410 to a desired depth corresponding to the depth of the cutting plane to be created.

In the case of a vertical cutting plane, the cutting plane may be located:
below the focal plane of the cutting apparatus in the case where the conical phase mask used allows the SLM to emulate a positive axicon (the Bessel ring 33a is located above the line of impact used to perform the cutting),
above the focal plane of the cutting apparatus in the case where the conical phase mask used allows the SLM to emulate a negative axicon (the Bessel ring 33a is located below the line of impact used to perform the cutting).

Preferably, the distance between two successive cutting planes is comprised between 2 μm and 500 μm, and in particular:
between 2 and 20 μm to treat a volume requiring high accuracy, for example in refractive surgery, preferably with a spacing comprised between 5 and 10 μm, or
between 20 and 500 μm to treat a volume not requiring high accuracy, such as to destroy the central part of a crystalline lens nucleus, preferably with a spacing comprised between 50 and 300 μm.

Of course, this distance can vary within a volume composed of a stack of cutting planes.

### 2.4. Control Unit

As indicated previously, the control unit 60 allows monitoring the various components of the cutting apparatus, namely the femtosecond laser source 10, the shaping system 30, the sweeping optical scanner 40 and the optical focusing system 50.

The control unit 60 is connected to these various components via one (or more) communication bus(es) allowing:
the transmission of control signals such as:
   the activation signal to the femtosecond laser source 10,
   the phase mask to the shaping system 30,
   the sweeping speed to the sweeping optical scanner 40,
   the position of the sweeping optical scanner 40 along the movement path,
   the cutting depth to the optical focusing system 50.
the receipt of measurement data derived from the various elements of the system such as:
   the sweeping speed reached by the optical scanner, or
   the position of the optical focusing system, etc.

The control unit 60 may be composed of one or more workstation(s) and/or one or more computer(s), or may be of any other type known to those skilled in the art. The control unit 60 may for example comprise a mobile phone, a tablet computer (such as an iPad®), a personal digital assistant (PDA), etc.

In all cases, the control unit 60 comprises a processor programmed to allow the driving of the femtosecond laser source 10, of the shaping system 30, of the sweeping optical scanner 40, of the optical focusing system 50, etc.

Advantageously, the control unit 60 is programmed to vary the shape of the modulated laser beam between two successive cutting planes, in particular between a horizontal cutting plane and a vertical cutting plane.

### 2.5. Operating Principle

The operating principle of the cutting device according to the invention will now be described in more detail by detailing:
the operation of the apparatus to create a horizontal cutting plane,
the operation of the apparatus to create a vertical cutting plane,
the operation of the apparatus to create a superposition of stacked horizontal and vertical cutting planes.

### 2.5.1. Formation of a Horizontal Cutting Plane

Within the framework of the present invention, the formation of a horizontal cutting plane is performed as follows.

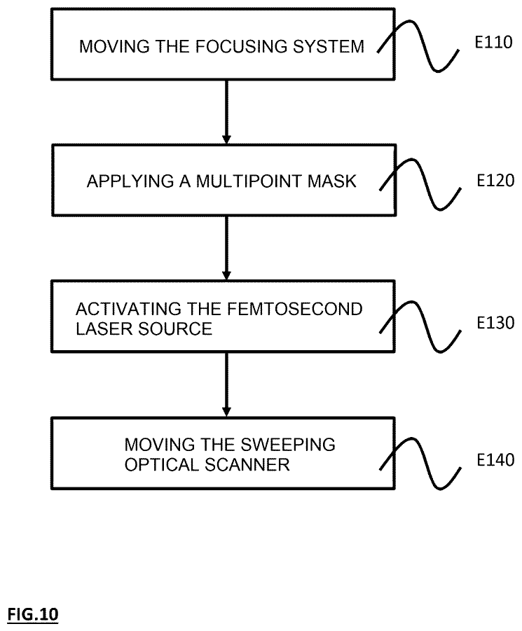

Referring to FIG. 10, the control unit 60 emits a control signal to the optical focusing system 50 to drive its movement so as to make the focal plane of the cutting apparatus coincide with the desired horizontal cutting plane (step E110).

The control unit 60 transmits a multipoint phase mask to the shaping system 30 to produce a multipoint modulated laser beam (step E120).

The control unit 60 also activates the movement of the sweeping optical scanner 40 to an initial position of the first sweeping optical path. Since the sweeping is carried out in X, Y, the scanner is equipped with one or more mirrors. For example, in the embodiment illustrated in FIG. 5, the sweeping optical scanner 40 includes a first mirror X and a second mirror Y whose pivoting allows moving the modulated laser beam along the first movement path. As a variant, the scanner may be equipped with a single mirror configured to pivot about two distinct axes.

When:
the focusing system 50 and the optical scanner 40 are in position (i.e. the scanner has reached a target start-of-line position),
the multipoint phase mask is loaded into the shaping system 30, and
the pivoting speed of the mirror(s) of the optical scanner 40 is constant,

The control unit 60 activates the femtosecond laser source 10 to emit a laser pulse (step E130). The femtosecond laser source 10 generates a laser beam 110 that passes through the shaping system 30. The shaping system 30 modulates the phase of the laser beam to produce a single multipoint modulated laser beam.

The multipoint modulated laser beam 310 exits the shaping system 30 and enters the optical scanner 40, which deflects the multipoint modulated laser beam 310.

The modulated and deflected laser beam 410 enters the optical focusing system 50, which focuses the beam in the focal plane of the cutting apparatus. In the focal plane, the modulation setpoint (i.e., the multipoint phase mask) applied to the shaping system 30 allows distributing the energy into a plurality of impact points. This plurality of simultaneously generated impact points forms the pattern. Each impact point of the pattern simultaneously produces a gas bubble.

The femtosecond laser 10 continues to emit other pulses in the form of a laser beam at a determined rate. Between each pulse, the mirror(s) pivot(s) by a certain angle, which results in moving the pattern 8 and producing new gas bubbles offset relative to the previous ones along the first optical path (step E140).

The operations of driving the femtosecond laser source 10 and the sweeping optical scanner 40 are repeated to form the horizontal cutting plane.

By varying the movement speed of the mirror(s) and/or the generation rate of the pulses by the femtosecond laser source 10, it is possible to vary the distance between two successive patterns.

Once the cutting line has been completed, the control unit 60 deactivates the femtosecond laser source 10 and controls the movement of the optical scanner 40 to a next cutting position according to the first movement path.

When the optical scanner 40 is in position and the mirror(s) has/have reached is/their constant setpoint speed, the control unit 60 reactivates the femtosecond laser source 10. The laser beam 110 passes through the shaping system 30, the optical scanner 40 and the optical focusing system 50. A new line of gas bubbles—parallel to the previous one—forms in the cutting plane.

When the optical scanner 40 has swept all the positions of the first movement path, the horizontal cutting plane is finalized.

In summary, for the creation of a horizontal cutting plane, the control unit 60 is configured to:
apply a multipoint phase mask to the shaping system 30 to produce a single multipoint modulated laser beam, the multipoint phase mask being calculated to distribute the energy of the multipoint modulated laser beam into at least two impact points in the focal plane of the cutting apparatus,
control the movement of the focusing system 50 to make the focal plane of the cutting apparatus coincide with the desired depth for the horizontal cutting plane, drive the sweeping optical scanner 40 to move the impact points of the single multipoint modulated laser beam along a first movement path, and
activate the femtosecond laser source 10 (after stabilization of the movement speed of the scanner).

### 2.5.2. Formation of a Vertical Cutting Plane

Within the framework of the present invention, the formation of a vertical cutting plane is performed as follows.

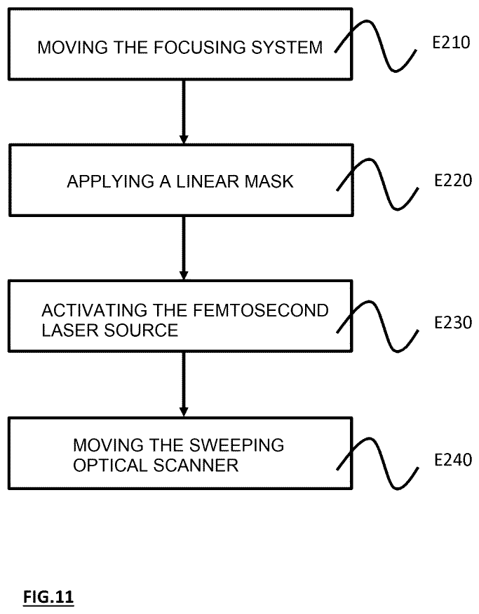

Referring to FIG. 11, the control unit 60 emits a control signal to the optical focusing system 50 to drive its movement so as to position the focal plane of the cutting apparatus at a given depth relative to the desired position for the vertical cutting plane (step E210). Indeed, as indicated previously, in the case of a vertical cutting plane, the cutting plane can be located:
above the focal plane of the cutting apparatus if the conical phase mask used allows the SLM to emulate a positive axicon (the Bessel ring 33a is located above the line of impact used to perform the cutting), in this case the control unit 60 drives the optical focusing system 50 to focus the modulated and deflected laser beam 410 to a desired depth smaller than the maximum depth of the vertical cutting plane to be created,
below the focal plane of the cutting apparatus in the case where the conical phase mask used allows the SLM to emulate a negative axicon (the Bessel ring 33a is located below the line of impact used to perform the cutting), in this case the control unit 60 drives the optical focusing system 50 to focus the modulated and deflected laser beam 410 to a desired depth greater than the maximum depth of the cutting plane to be created.

The control unit 60 transmits a conical phase mask (i.e. axicon modulation setpoint) to the shaping system 30 to produce a Bessel modulated laser beam (step E220).

The control unit 60 also activates the movement of the sweeping optical scanner 40 to an initial position of the second optical scanning path.

When:
the focusing system 50 and the optical scanner 40 are in position,
the conical phase mask is loaded into the shaping system 30, and
the pivoting speed of the mirror(s) of the optical scanner 40 is constant,

The control unit 60 activates the femtosecond laser source 10 to emit a laser pulse (step E230). The femtosecond laser source 10 generates a laser beam 110 that passes through the shaping system 30. The shaping system 30 modulates the phase of the laser beam to produce a single Bessel-type modulated laser beam.

The Bessel-type modulated laser beam 310 exits the shaping system 30 and enters the optical scanner 40 which deflects the Bessel-type modulated laser beam 310.

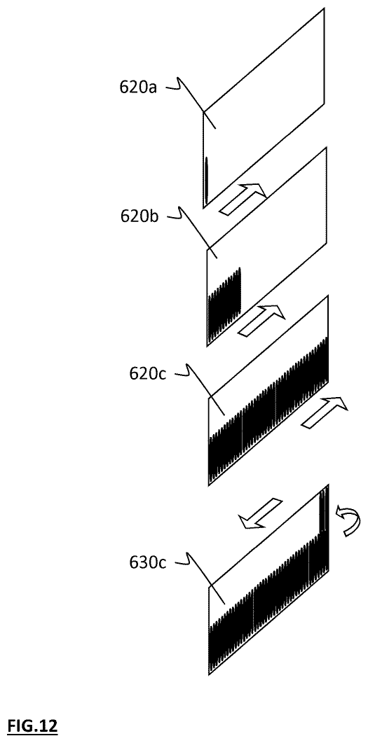

The modulated and deflected laser beam 410 enters the optical focusing system 50 which focuses the beam. Above or below the focal plane, the modulation setpoint (i.e., the conical phase mask) applied to the shaping system 30 allows distributing the energy into a line of impact. This line of impact produces an oblong gas bubble extending parallel to the optical axis A-A′ of the cutting apparatus, as illustrated in step 620a of FIG. 12.

The femtosecond laser 1 continues to emit other pulses in the form of a laser beam at a determined rate. Between each pulse, the mirror(s) pivot(s) by a certain angle, which has the consequence of moving the line of impact and forming a new oblong gas bubble along the second optical path (step E240). This new gas bubble is adjacent to the previously formed oblong gas bubble, as illustrated in step 620b of FIG. 12 after a certain number of pulses, thus forming a vertical cutting plane segment.

The operations of driving the femtosecond laser source 10 and the sweeping optical scanner 40 are repeated to form the vertical cutting plane.

More specifically, the pivoting of the mirror(s) between each pulse of the femtosecond laser source 10 has the consequence of moving the line of impact and producing new oblong gas bubbles offset relative to the previous ones, until forming a cutting section in the cutting plane, as illustrated in step 620c of FIG. 12.

Once a cutting section has been completed, the control unit 60 deactivates the femtosecond laser source 10, and controls the movement of the optical focusing system for the creation of a second section of oblong gas bubbles above the first section, then again controls the restart of the pivoting of the mirror(s) in the opposite direction and activates the femtosecond laser source 10 again, as illustrated in step 630c of FIG. 12.

The laser beam 110 passes through the shaping system 30, the optical scanner 40 and the optical focusing system 50. A new section of oblong gas bubbles—located above the previous section and extending in the same plane as the previous section—forms in the cutting plane. When the optical scanner 40 has swept all the positions of the second movement path, the vertical cutting plane is complete.

In summary, for the creation of a vertical cutting plane, the control unit 60 is configured to:
apply an axicon modulation setpoint to the shaping system 30 in order to produce a Bessel-type modulated laser beam from the Gaussian laser beam, said modulation setpoint including a phase mask 314, 315 emulating an axicon applied to the spatial light modulator (SLM), said Bessel-type modulated laser beam creating a line of impact that allows generating an oblong gas bubble in the tissue,
drive the sweeping optical scanner 40 to move the line of impact of the Bessel-type modulated laser beam along a second optical movement path to form a vertical plane consisting of a set of adjacent oblong gas bubbles.

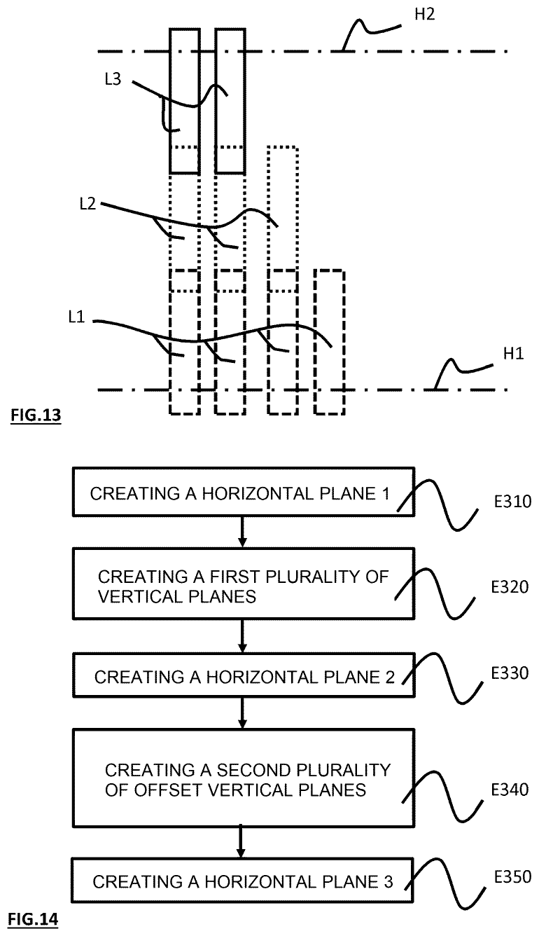

Referring to FIG. 13, a set of lines of impact L1, L2 and L3 used to create stacked oblong gas bubble sections extending in a single vertical cutting plane located between two horizontal cutting planes H1, H2, is partially illustrated.

As shown in FIG. 13, the lines of impact used to form two successive stacked sections partially overlap. Indeed, the inventors discovered that the end portions of a line of impact do not have sufficient energy to form the oblong gas bubble. Therefore, the inventors propose an overlap of the lines of impact used to create stacked oblong gas bubble sections.

Particularly, after the creation of a horizontal cutting plane H1, the vertical cutting planes located above the horizontal cutting plane H1 are created by stacking successive oblong gas bubble sections, for example first, second and third sections in the embodiment illustrated in FIG. 13.

For the creation of the first section (i.e. the section closest to the horizontal cutting plane H1), the control unit 60 is configured to drive the optical focusing system 50 in order to position the focal plane of the cutting apparatus at a predefined non-zero distance from the first horizontal cutting plane. This predefined distance is smaller than the length of the line of impact L1 of the Bessel-type modulated laser beam. In particular, the predefined distance may be comprised between ⅕ and ⅓ of the length of the line of impact. Thus, each line of impact L1 used for the creation of the first section partially intersects the horizontal cutting plane H1. The control unit 60 then drives the sweeping optical scanner 40 to move the line of impact L1 along the second optical movement path to form the first section of adjacent oblong gas bubbles.

For the creation of the second section (i.e., the section located above the first section), the control unit 60 is configured to drive the optical focusing system 50 in order to position the focal plane of the cutting apparatus at the predefined distance from the first section of oblong gas bubbles. Thus, each line of impact L2 used for the creation of the second section partially covers the oblong gas bubbles of the first section. The control unit 60 then drives the sweeping optical scanner 40 to move the line of impact L2 along the second optical movement path to form the second section of adjacent oblong gas bubbles.

For the creation of the third section (i.e., the section furthest from the horizontal cutting plane H1), the control unit 60 is configured to drive the optical focusing system 50 in order to position the focal plane of the cutting apparatus at the predefined distance from the second section so that an end portion of the line of impact L3 is in contact with the oblong gas bubbles of the second section.

This guarantees that the tissue bridges between the different stacked sections are narrow enough to ensure dissection by the practitioner of acceptable quality over the entire height of each vertical cutting plane.

### 2.5.3. Formation of Horizontal and Vertical Cutting Planes Stacked to Create Tissue Cubes

The operating principle of the cutting apparatus with reference to the destruction of a crystalline lens as part of a cataract surgery will now be described in more detail.

To partition the crystalline lens into cubes that could be suctioned by a suction cannula, horizontal and vertical cutting planes are formed by starting with the deepest horizontal cutting plane in the crystalline lens and by stacking the successive vertical and horizontal cutting planes up to the most superficial horizontal cutting plane in the crystalline lens.

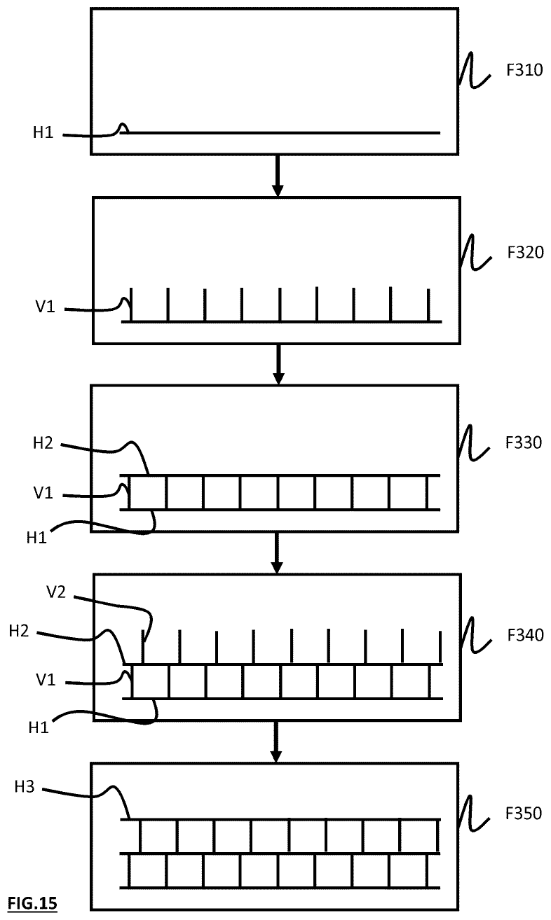

Referring to FIGS. 14 and 15, the deepest horizontal cutting plane H1 is created in a first step (E310, F310). The control unit 60:
applies a multipoint phase mask to the shaping system 30 to produce a multipoint modulated laser beam,
controls the movement of the focusing system 50 to make the focal plane of the cutting apparatus coincide with the desired deepest cutting plane,
drives the movement of the sweeping optical scanner to a target position along the first optical path (for example in crenellation), and
activates the femtosecond laser source 10 (after stabilizing the scanner movement speed and reaching the target position).

A series of shots is performed in the focal plane of the cutting apparatus. With each shot, several impact points simultaneously focus in the focal plane. Each impact point forms a gas bubble. The optical scanner allows moving the multiple impact points in the focal plane between each shot. When the entire surface of the horizontal cutting plane is covered with gas bubbles, the horizontal cutting plane H1 is complete.

In a second step (E320 and F320), a first set of adjacent vertical cutting planes V1 is created with the cutting apparatus. For each vertical cutting plane V1, the control unit 60:
applies a conical phase mask to the shaping system 30 to produce a Bessel modulated laser beam,
controls the movement of the focusing system 50 to position the line of impact in the cutting plane (the focusing plane being above or below the cutting plane depending on whether the axicon emulated on the shaping system is a positive or negative axicon),
drives the movement of the sweeping optical scanner to a target position along the second optical path (for example a segment), and
activates the femtosecond laser source 10 (after stabilizing the scanner movement speed and reaching the target position).

A series of shots is performed. With each shot, a line of impact is generated. Each line of impact forms an oblong gas bubble along the optical axis of propagation of the modulated laser beam. The sweeping optical scanner 40 allows moving the line of impact above/below the focal plane between each shot. When the entire second movement path is covered with gas bubbles, the vertical cutting plane V1 is complete.

If the depth of the line of impact is smaller than the desired depth for the vertical cutting plane, then the control unit 60 can monitor the sweeping optical scanner 40 and the optical focusing system 50 to move the line of impact along the second optical path by varying the depth of the focal plane of the cutting apparatus for the round trip.

Several vertical cutting planes V1 above the initial horizontal cutting plane H1 are thus obtained. These cutting planes V1 are divided into two subgroups (a first subgroup of planes 107′ and a second subgroup of planes 107″ in FIG. 2) forming parallel planes within the same subgroup but perpendicular for 2 different subgroups thus making it possible to obtain vertical cutting planes forming a grid pattern, each elementary square representing the side walls of the cubes thus cut.

In a third step (E330, F330), an intermediate horizontal plane H2 is created to cover the vertical cutting planes V1. This horizontal cutting plane H2 is created according to the same method as the one described with reference to the first step. Crystalline cubes defined between the horizontal and vertical planes created in the first, second and third steps are thus obtained. In a fourth step (E340, F340), a second set of adjacent vertical cutting planes V2 is created with the cutting apparatus. This second set of vertical cutting planes V2 is laterally offset relative to the first set of vertical cutting planes V1. To do so, the control unit 60 drives the movement of the sweeping optical scanner 40 along a different third movement path—particularly laterally offset—relative to the second optical path. Thus, none of the vertical cutting planes V2 of the second set is coplanar with a vertical cutting plane V1 of the first set. In a fifth step (E350, F350), an upper horizontal plane H3 is created using the same method as the one described with reference to the first step. A stack of two stages of crystalline lens cubes is thus obtained, the crystalline lens cubes of the second stage being laterally offset relative to the crystalline lens cubes of the first stage.

The preceding steps can be repeated to perform a stack of more than two stages of crystalline lens cubes, the cubes of one stage being laterally offset from the crystalline lens cubes located on the lower stage on which they rest.

### 3. CONCLUSIONS

The lateral offset of the vertical cutting planes located above a horizontal cutting plane relative to the vertical cutting planes located below said horizontal cutting plane allows limiting the propagation of the gas bubbles to the surface of the tissue to be treated.

As previously indicated, in the case of ocular tissue cutting, such propagation can cause gas accumulation above the cutting plane in the crystalline lens, or even above the crystalline lens in the anterior chamber of the eye, and mask the laser beam, thereby preventing the cutting in the anterior part of the crystalline lens.

Thus, the invention provides an efficient three-dimensional cutting tool that allows making cuts into elementary portions of the same size and small dimensions.

The reader will understand that numerous modifications can be made to the invention described above without materially departing from the new teachings and advantages described herein.

1. A cutting apparatus for cutting a tissue, said apparatus including a femtosecond laser source configured to emit a Gaussian laser beam in the form of pulses and a processing device for processing the Gaussian laser beam, the processing device being disposed downstream of the femtosecond laser source, the processing device comprising:
a shaping system positioned on the trajectory of the Gaussian laser beam, to modulate the phase of the wavefront of the Gaussian laser beam, the shaping system comprising a spatial light modulator and being configured to produce a modulated laser beam from the Gaussian laser beam,
a sweeping optical scanner disposed downstream of the shaping system to move the modulated laser beam,
an optical focusing system downstream of the shaping system, configured to focus the modulated laser beam in a focal plane of the cutting apparatus and configured to move the focal plane of the cutting apparatus into a plurality of positions along an optical axis of propagation of the modulated laser beam,
wherein the processing device further comprises a control unit to drive the femtosecond laser source, the shaping system, the sweeping optical scanner and the optical focusing system in order to create successive horizontal and vertical cutting planes, the horizontal cutting planes extending perpendicularly to the optical axis and the vertical cutting planes extending parallel to the optical axis, said control unit being configured to:
control the creation of a first horizontal cutting plane,
control the creation of a first plurality of vertical cutting planes above the first horizontal cutting plane,
control the creation of a second horizontal cutting plane above the first plurality of vertical cutting planes, the first horizontal cutting plane being deeper in the tissue than the second horizontal cutting plane,
control the creation of a second plurality of vertical planes above the second horizontal plane, wherein the second plurality of vertical cutting planes is laterally offset relative to the first plurality of vertical planes.

2. The cutting apparatus according to claim 1, wherein the second plurality of vertical cutting planes is laterally offset relative to the first plurality of vertical cutting planes by a distance comprised between 5 μm and 500 μm.

3. The cutting apparatus according to claim 1, wherein the second plurality of vertical cutting planes is laterally offset relative to the first plurality of vertical cutting planes along first and second axes perpendicular to the optical axis, the first and second axes being orthogonal to each other.

4. The cutting apparatus according to claim 1, wherein, for the creation of each horizontal cutting plane, the control unit is configured to:
apply a multipoint phase mask to the shaping system to produce a single multipoint modulated laser beam, the multipoint phase mask being calculated to distribute the energy of the multipoint modulated laser beam into at least two impact points in the focal plane of the cutting apparatus,
control the movement of the focusing system to make the focal plane of the cutting apparatus coincide with the desired depth for the horizontal cutting plane,
drive the sweeping optical scanner to move the impact points of the single multipoint modulated laser beam along a first movement path,
activate the femtosecond laser source.

5. The cutting apparatus according to claim 1, wherein for the creation of each vertical cutting plane of the first plurality, the control unit is configured to:
apply an axicon modulation setpoint to the shaping system in order to produce a Bessel-type modulated laser beam from the Gaussian laser beam, said modulation setpoint including a phase mask emulating an axicon applied to the spatial light modulator, said Bessel-type modulated laser beam creating an line of impact that allows generating an oblong gas bubble in the tissue,
drive the sweeping optical scanner to move the line of impact of the Bessel-type modulated laser beam along a second optical movement path to form a set of adjacent oblong gas bubbles.

6. The cutting apparatus according to claim 5, wherein for the creation of each vertical cutting plane of the second plurality, the control unit is configured to:
apply the axicon modulation setpoint to the shaping system,
drive the sweeping optical scanner to move the line of impact of the Bessel-type modulated laser beam along a third optical path laterally offset relative to the second optical path.

7. The cutting apparatus according to claim 5, wherein each vertical cutting plane is composed of a stack of several sets of adjacent oblong gas bubbles, the control unit being configured to:
drive the optical focusing system in order to position the focal plane of the cutting apparatus at a predefined non-zero distance from the first horizontal cutting plane, said predefined distance being smaller than the length of the line of impact of the Bessel-type modulated laser beam such that the line of impact partially intersects the first horizontal cutting plane,
drive the sweeping optical scanner to move the line of impact of the Bessel-type modulated laser beam to form a first set of adjacent oblong gas bubbles,
drive the optical focusing system in order to position the focal plane of the cutting apparatus at the predefined distance from the first set of adjacent oblong gas bubbles such that the line of impact partially intersects the first set of adjacent oblong gas bubbles,
drive the sweeping optical scanner to move the line of impact of the Bessel-type modulated laser beam to form a second set of adjacent oblong gas bubbles.

8. The cutting apparatus according to claim 7, wherein the predefined distance is comprised between ⅕ and ⅓ of the length of the line of impact.
